SEQUENCE LISTING (8 sequences)

SEQ ID NO: 1 - protein, 31 aa, Artificial Sequence
  Consensus sequence of WW domain from various organisms
LXXGWXXXXXXXGXXXXXBHXXXTXXWXXPX

SEQ ID NO: 2 - DNA, 31 bp, Artificial Sequence
  PCR forward primer containing restriction enzyme linker sequence ABA link F
cgggatccatgaattctcttagtgggactta

SEQ ID NO: 3 - DNA, 32 bp, Artificial Sequence
  PCR reverse primer containing restriction enzyme linker sequence ABA link R2
ctagtctagatgcagtcaacttttccaagaac

SEQ ID NO: 4 - protein, 26 aa, Barley ABAP1
WTEHTSPEGFKYYYNSITRESKWEKP

SEQ ID NO: 5 - protein, 26 aa, Arabidopsis FCA
WTEHTSPDGFKYYYNGLTGESKWEKP

SEQ ID NO: 6 - protein, 26 aa, Human FBP
WVEGITSEGYHYYYDLISGASQWEKP

SEQ ID NO: 7 - protein, 26 aa, Mice FBP
WTEHKSPDGRTYYYNTETKQSTWEKP

SEQ ID NO: 8 - protein, 4 aa, Arabidopsis FCA
PPLP

(19) United States
(12) Patent Application Publication
(10) Pub. No.: US 2006/0179518 A1
(43) Pub. Date: Aug. 10, 2006

(54) Plant proteins having an abscisic acid binding site and methods of use

(75) Inventors: Robert Hill, Winnipeg (CA); Fawzi A. Razem, Winnipeg (CA); Ashraf El-Kereamy, Winnipeg (CA); Santosh Kumar, Winnipeg (CA)

Correspondence Address: BIRCH STEWART KOLASCH & BIRCH, PO BOX 747, FALLS CHURCH, VA, 22040-0747, US

(73) Assignee: The University of Manitoba, Winnipeg (CA)

(21) Appl. No.: 11/297,427

(22) Filed: Dec. 9, 2005

Related U.S. Application Data
(60) Provisional application No. 60/634,435, filed on Dec. 9, 2004.

(51) Int. Cl. A01H 1/00; C12N 15/82; C07K 14/415

(52) U.S. Cl. 800/287; 530/370

(57) ABSTRACT

Proteins having binding sites for abscisic acid (ABA) and methods of use are disclosed. The physiological functions of ABA, plant life cycles, seed dormancy and ripening can be altered by manipulating the binding of ABA to its receptors.

### FIELD OF THE INVENTION

The present invention relates generally to plant proteins involved in signal transduction. More particularly, the present invention relates to proteins having an abscisic acid binding site, methods to isolate proteins having an abscisic acid binding site, and methods to manipulate the effects of abscisic acid in plants.

### BACKGROUND OF THE INVENTION

Transition to flowering is a critical developmental step in the life cycle of plants and is controlled by multiple regulatory genes. The transition to flowering occurs through highly coordinated processes and requires the integration of multiple regulatory pathways^{A-G}. For example, several plants utilize long days and cold temperature as environmental sensors of seasonal progression^{G,H} and gibberellic acid (hereinafter “GA”) as a developmental indicator^{I}. These regulatory pathways are also involved in the control of the time of flowering through a coordinated interaction between the endogenous developmental factors and the surrounding environmental cues^{D}.

Following flowering, further regulatory pathways are activated or inhibited to permit seed ripening, dessication, and seed dispersal. In the production of certain crops, it is necessary that the seeds be fully ripe prior to harvesting in order to achieve optimal characteristics of any product that is produced from the seed. For example, in the production of canola oil, failure to complete seed ripening of the canola crop generally results in lower oil quality due to the presence of chlorophyll within the seed, even when the seed is treated with dessicants.

Similarly, seed dormancy periods are highly regulated by pathways that respond to various environmental stress factors, for example drought or salt exposure. Dormant periods are characterized by cessation of growth or development and the suspension of metabolic processes.

In the field of stress responses, certain advances have been made in determining the plant proteins and regulatory pathways responsible for adaptation to stress conditions, and as a result, plants can now be genetically engineered to withstand a greater degree of environmental stresses, and to quickly recover and re-initiate the reproductive cycle following periods of stress.

Flowering Control

With respect to transition to flowering, the Arabidopsis FCA (flowering control protein) gene is amongst the most studied of the identified flowering genes. It encodes an RNA-binding protein (FCA protein), which promotes flowering through repression of Flowering locus C (FLC). The FLC gene is otherwise expressed to FLC protein, which is a transcription factor that promotes the transcription of genes to prevent flowering.

FLC represents a convergence point for several flowering time regulatory pathways, including autonomous and vernalization. An autonomous pathway that is suggested to be independent of environmental cues, controls the expression level of FLC, while promotion of flowering through FLC repression occurs during vernalization as a result of prolonged exposure to cold^{N}.

Genetic analyses of flowering time control have identified many of the components involved in these regulatory pathways^{A}. At least six genes have been identified in the autonomous pathway, all of which operate in separate but parallel pathways to regulate FLC expression^{A,B,D}. One of these genes, FCA, encodes FCA protein, which possesses RNA binding domains and a WW protein interaction domain^{O}. The FCA floral promotion gene has been cloned and shown to contain 20 introns^{O}. The alternative splicing of FCA pre-mRNA introns 3 and 13 produces four distinct transcripts, one of which, FCAγ, has all its introns accurately spliced and removed and has been shown to promote flowering^{O}. Another major, but inactive transcript, FCAβ, is generated as a result of cleavage and polyadenylation within intron 3^{P}. This selection for active and/or inactive FCA transcripts is developmentally regulated^{P,Q}. Recent studies have shown that the FCA protein is negatively regulating its own expression by promoting cleavage and polyadenylation within intron 3^{R}, with the result that inactive FCAβ transcript will accumulate at the expense of functional FCAγ. Quesada et al.^{R} have shown that this negative regulation requires the FCA WW protein interaction domain. Subsequent studies have identified the interactor to be the polyadenylation factor, FY, through its Pro-Pro-Leu-Pro sequence^{S}. Following interaction of FCA WW with FY, it is suggested that the complex (i.e., FCA-FY) binds to FCA pre-mRNA, thus blocking processing of active FCAγ mRNA transcripts and promoting the expression of inactive FCAβ^{Q}.

FCA is constitutively expressed throughout plant development. The fca mutation, for example, affects multiple phases of plant development, an indication that FCA is required throughout plant development, in agreement with the virtually equal FCAγ expression levels reported in different plant organs^{O}.

Thus, FCA must bind the polyadenylation factor, FY, at its WW protein interaction domain, to autoregulate its mRNA and repress FLC, resulting in flowering.

Gibberellic acid, a developmental indicator, has been shown to be involved in flowering time control, however, this is the only growth regulator that has been suggested to play a role in flowering time control.

Abscisic Acid

The plant hormone abscisic acid (hereinafter “ABA”) regulates various physiological processes in plant development and is a key hormone in plant abiotic stress responses. These roles include agronomically important processes, such as its involvement in seed dormancy, synthesis of storage proteins, and lipid accumulation and its mediation of stress-induced processes (1-3). Following perception of ABA by plant cells, the cellular responses can be either very quick, such as ion channeling in guard cells, or slow and require changes in gene expression (4). In both situations, it is assumed that cellular response to ABA requires some kind of interaction between ABA molecules and receptors followed by protein phosphorylation that finally target the transcription of genes involved in stress-induced processes (4, 5).

Certain ABA mutants (e.g., 6, 7) have been identified, having different responses to ABA, and the molecular mechanism underlying ABA perception is still poorly understood. For example, in high-mountain potatoes, exogenously applied ABA favors tuberization whereas gibberellic acid favors flowering^{X}. In addition, the ABA-deficient mutants of Arabidopsis in addition to a dwarf habit, flower early^{C}. There has been no success in characterizing putative ABA receptors even with the use of genetic approaches (4).

High-affinity binding sites for ABA have been reported, however, in membrane fractions and guard cell plasmalemma of Vicia faba (8), microsomal fractions from Arabidopsis thaliana (9), the cytosol of the developing flesh of apple fruits (10) and more recently, an ABA-specific binding site was purified from the epidermis of broad bean leaves (11). The site of ABA perception has also been located at the extracellular side of the plasma membrane of barley aleurone tissue. However, due to difficulties in purifying ABA-binding proteins, most studies on ABA binding were carried out by either using total protein extracts or histochemical probes. Furthermore, it has always been difficult to relate these proteins to any physiological role of ABA in plants (4, 12).

Despite numerous attempts to isolate membrane-bound hormone receptors in plants, little progress has been made in identifying ABA receptors owing to their low abundance relative to other proteins in plant cells. One approach to identify a putative ABA receptor is to clone and characterize an ABA-binding protein (5). Anti-idiotypic antibodies (AB2) have been used to identify and isolate animal hormone receptors (18) and to clone an ABA-induced gene in barley aleurone (19).

It is, therefore, desirable to determine the mechanism by which abscisic acid correlates with plant abiotic stress responses, and to determine other plant processes that may rely on the presence of abscisic acid.

It is also desirable to identify proteins capable of binding abscisic acid and to determine whether a common binding site exists between various abscisic acid receptors.

It is further desirable to characterize the abscisic acid binding site in order to enable targeting or alteration of the binding site such that abscisic acid effects can be manipulated as necessary to elicit desirable effects in the plant, and to develop activators and inhibitors for manipulating certain functions of abscisic acid.

### SUMMARY OF THE INVENTION

In accordance with the invention, there is provided a method of regulating the expression of proteins in seed development including the step of introducing an effective amount of ABAP1 or an operative fragment thereof into a developing seed with or without ABA.

In accordance with an alternate embodiment, there is provided a method of regulating seed germination comprising the step of introducing an effective amount of ABAP1 or an operative fragment thereof into a seed with or without ABA.

The invention also provides a method for synergistically regulating the expression of proteins in seed development comprising the step of introducing an effective amount of ABAP1 or an operative fragment thereof and abscisic acid (ABA) into a developing seed.

Still further, the invention provides an ABAP1 fragment retaining abscisic acid (ABA) binding capability wherein the fragment is 10 kDa or larger characterized by a hydrophobic region HR2 or 21 kDa or larger characterized by two hydrophobic regions, HR1 and HR2.

In yet another embodiment, the invention provides a method of modulating abscisic acid (ABA)-mediated signal transduction comprising the step of introducing an effective amount of ABAP1 or an operative fragment thereof or ABA or mixtures thereof to inhibit or promote plant flowering.

Further still, the invention provides a method of isolating and purifying ABAP1 comprising the steps of: infecting a recombinant clone; inducing over expression of ABAP1; and, isolating and purifying ABAP 1.

### BRIEF DESCRIPTION OF THE DRAWINGS

Embodiments of the present invention will now be described, by way of example only, with reference to the attached Figures, wherein:

FIG. 1 is a comparison of the WW domain sequence between Barley ABAP1, Arabidopsis FCA, Human FBP, and Mouse FBP;

FIG. 2a is a Southern blot analysis of genomic DNA of various plants following digestion by BamH1;

FIG. 2b is a Northern blot analysis of ABAP1 mRNA from barley embryo, leaves, and aleurone;

FIG. 3 are graphs of the binding of ³H⁺-ABA to ABAP1 relative to denatured ABAP1 and BSA (A) and at varying pH (B);

FIG. 4 are graphs of the association and dissociation kinetics of ABA binding to ABAP1;

FIG. 5 are graphs of the saturation binding of ³H⁺-ABA to ABAP1;

FIG. 6 are graphs of the displacement of ³H⁺-ABA by ABA analogs and precursors;

FIG. 7 is a structural representation of ABAP1 and fragments, in accordance with various embodiments of the invention;

FIG. 8a is a hydrophobicity analysis of ABAP1 showing the relative location of the HR1 and HR2 domains;

FIG. 8b is a structural representation of ABAP1 and its fragments after trypsin digestion;

FIG. 8c is a graph of ABA binding activity of ABAP1 and its fragments;

FIG. 9 is a graph of GUS activity after treatment with eₘ and eₘ with ABAP1;

FIG. 10 is a graph of the effects of competitive inhibitors of ABA on eₘ promoter activation by ABAP1;

FIG. 10a is a graph summarizing the effects of ABAP1, ABA and PBI51 in varying combinations on GUS activity;

FIG. 11 is a graph showing the negative effect of ABAP1 on α-amylase activity;

FIG. 11a is a graph showing the effect of ABAP1 on amylase activity at varying concentrations of ABA;

FIG. 11b is a graph summarizing the effects of ABAP1, ABA and PBI51 in varying combinations on amylase activity;

FIGS. 12 a-c are graphs and photographs showing the effects of ABAP1 on germination rates of McLeod barley embryos;

FIG. 13 is a graph showing the effects of ABAP1 on plumule growth rates of Harrington barley embryos;

FIG. 13a is a graph showing the effect of ABAP1 on radical growth rates of Harrington barley embryos;

FIG. 13b is a graph showing the effect of ABAP1 on germination rates at varying concentrations of ABA;

FIG. 14 is a structural representation comparing FCA and ABAP1;

FIG. 15 is a schematic diagram illustrating the mechanism known in the prior art by which FCA protein binds FY to permit translation of FLC protein, to permit flowering;

FIG. 16 are graphs showing the binding of ³H⁺-ABA to purified recombinant FCA Binding of ³H-(+)-ABA to the purified recombinant FCA protein. a, Binding of ³H-(+)-ABA by FCA. The incubation reactions contained different amounts of freshly prepared FCA protein in addition to 50 nM ³H-(+)-ABA and all buffer components as described in methods. b, Binding specificity. The incubation reactions contained either 10 μg freshly prepared FCA, 10 μg heat-denatured FCA, or 10 μg of BSA plus all buffer components as described in Methods. c, pH dependency. Assays contained all reaction components plus appropriate buffer adjusted to the pH values shown. The 100% binding activity corresponds to approximately 0.52 mol ABA mol⁻¹ protein. Each data point represents triplicate assays using three different protein purifications (error bars represent SD);

FIG. 17 are graphs showing the saturation kinetics of FCA protein binding to ABA- a, The FCA protein was incubated with increasing concentrations of ³H-(+)-ABA in the absence of (total binding) or in the presence of 5 μM unlabelled (+)-ABA (non-specific binding). Specific binding (SB) is shown (upper curve) and represents the difference between total and non-specific binding measurement (lower line). b, Scatchard analysis of the saturation ABA binding. All points fitted a linear relationship with r²=0.88 (r²=0.93 without the first point) and maximum binding was calculated 0.72 mol mol⁻¹ protein and the K_{d}=19 nM;

FIG. 18 is a graph showing ABA binding to FCA in the presence of ³H-(+)-ABA by (−)-ABA and trans-ABA analogs. The (+)-ABA was used as a control. All competition assays were carried out as described²²;

FIG. 19 illustrates the interference of (+)-ABA in FCA/FY interaction. To test ABA interference, FCA was bound with ABA for 30 min and the interaction between FCA/FY was carried out in the presence of either (−)- or (+)-ABA in binding buffer. Released proteins were separated on SDS-PAGE and labeled proteins were detected. FCA-WW-FY was used as a control (c)¹⁹. The 100% activity corresponds to highest DPM count observed for the control (approx. 2.5×10³). Concentration-dependent inhibition of FCA/FY interaction by ABA. The right panel shows ³⁵S activity and the 100% represents the highest DPM count (approx. 2.1×10³) observed for the control;

FIG. 20 illustrates the role of WW domain in ABA binding. GST:FCA-WW-FY interaction mixture was incubated for 90 min before 1 μM ³H-(+)-ABA was added and the mixture pelleted, washed, and the dual activity for [³⁵S]met-FY and ³H-(+)-ABA were counted as described in methods. Time of incubation after ABA addition is shown and time 0 represents the GST:FCA-WW-FY activity before ABA addition. To test if the mutation in the WF domain can abolish ABA binding, FCA-WF protein was used and binding assays were carried out as above. The 100% binding activity represents approximately 0.5 mol ABA mol⁻¹ FCA protein (for ABA binding) and an estimated 0.63 mol FY mol⁻¹ FCA protein. The activity of [³⁵S]met-FY in the absence of ABA was similar to the control (time 0) at the time points shown and were not included in the figure. The FCA ³H-(+)-ABA binding activity in the absence of FY reached approximately 50% saturation at 15 min and approximately 95% saturation at 45 min. Each data point represents triplicate assays and error bars represent SD;

FIG. 21 is an immunoblot of subcellular protein fractions of barley aleurone layers using AB2 antibodies; and,

FIG. 22 shows SDS-PAGE results in respect of ABAP1 purification and immunodetection.

### DETAILED DESCRIPTION

Generally, the present invention describes proteins that are capable of binding abscisic acid, and methods for manipulating the effects of abscisic acid with respect to stress responses, germination, flowering, and seed dormancy in plants.

Specifically, an ABA binding protein (ABAP1) has been characterized that shares high homology with FCA proteins from various species. The ABA binding site has been identified to include two HR (hydrophobic) regions flanked by hydrophilic platforms. ABAP1 genes have been detected in diverse monocot and dicot species, including wheat, alfalfa, tobacco, mustard, white clover, garden pea, and oilseed rape. ABAP1 lacks significant homology with any other known protein sequence.

Further, it has been determined that FCA binds abscisic acid (ABA) with high affinity, that is stereospecific, and follows receptor saturation kinetics. The binding of ABA to FCA displaces FY from FCA in a time and concentration dependent manner.

The invention also provides a method to isolate and identify ABA binding proteins, and describes methods to activate and inhibit ABA-dependent processes such as flowering, germination, and seed ripening.

ABAP1 Protein

A barley grain protein, designated ABAP1, and encoded by a previously sequenced gene (Accession No. AF127388) was purified and shown to specifically bind ABA. ABAP1 protein is a 472 amino-acid polypeptide containing a WW protein interaction domain and is induced by ABA treatment in aleurone layers. Polyclonal anti-idiotypic ABA antibodies (AB2) cross-reacted with the purified ABAP1 and with a corresponding 52 kDa protein associated with membrane fractions of ABA-treated barley aleurones. ABAP1 lacks significant homology with any known protein sequence, however the ABAP1 genes have here been detected in diverse monocot and dicot species, including wheat, tobacco, alfalfa, garden pea, and oilseed rape.

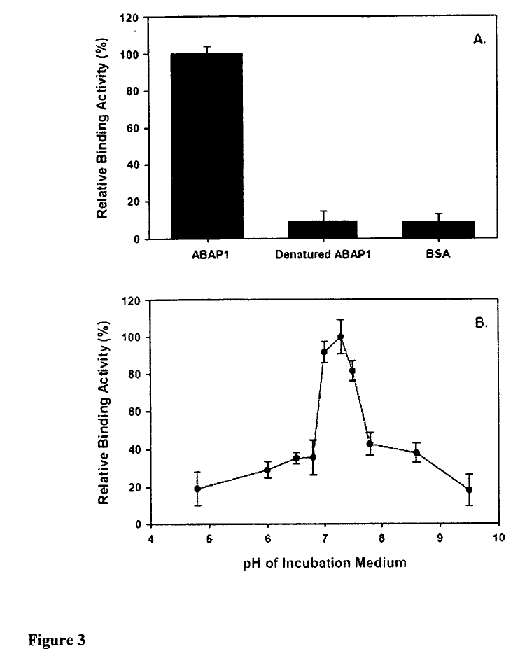

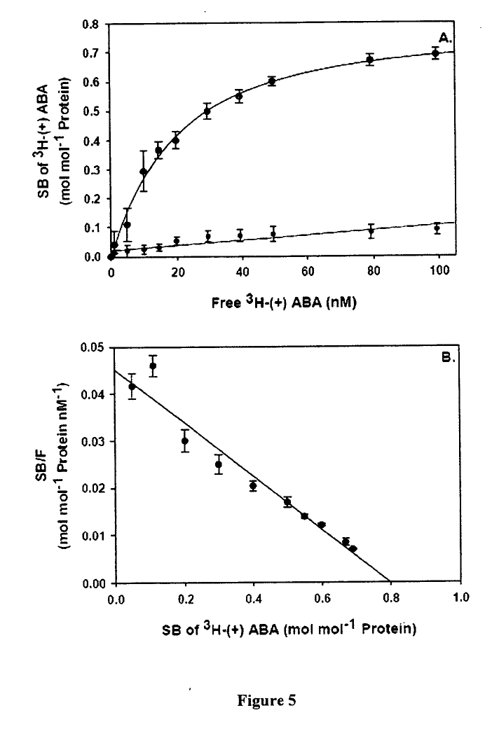

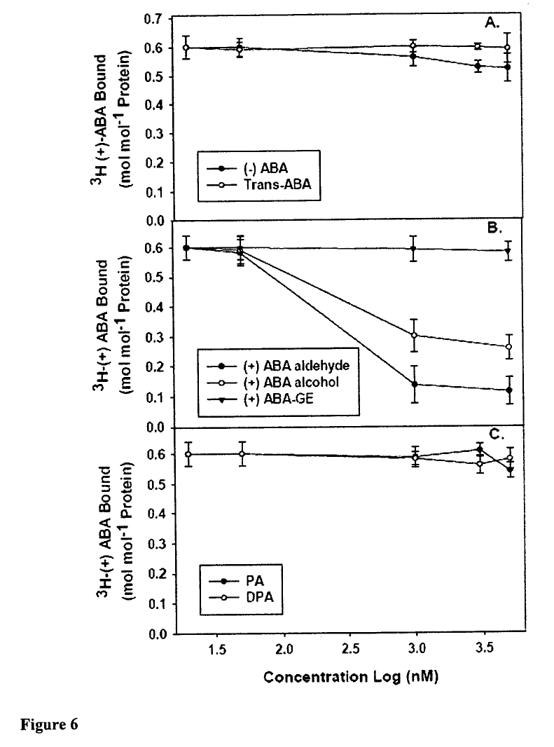

The recombinant ABAP1 protein bound ³H⁺-ABA optimally at a neutral pH. Denatured ABAP1 protein did not bind ³H⁺-ABA, nor did BSA (FIG. 3a). The maximum specific binding as shown by Scatchard plot analysis was 0.8 mol ABA mol⁻¹ protein with a linear function of r²=0.94, an indication of one ABA binding site with a dissociation constant of about K_{d}=28×10⁻⁹ M (FIG. 5). The stereospecificity of ABAP1 was established by the incapability of ABA analogs and metabolites including (−) ABA, trans-ABA, phaseic acid (PA), dihydrophaseic acid (DPA), and (+) abscisic acid-glucose ester (ABA-GE) to displace ³H⁺-ABA bound to ABAP1 (FIG. 6). Two ABA precursors, (+) ABA-aldehyde and (+) ABA-alcohol were, however, able to displace ³H⁺-ABA, an indication that the structural requirement of ABAP1 at C-1 position is not strict. Cumulatively, the data show that ABAP1 exerts high binding affinity for ABA. The interaction is reversible, follows saturation kinetics, and has stereospecificity, meeting the criteria for an ABA-binding protein.

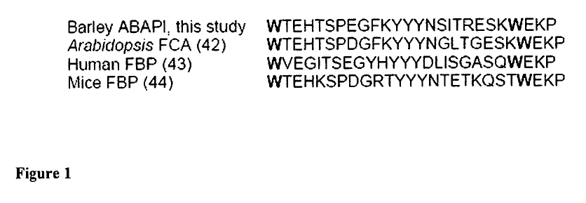

Hydrophobicity analysis of the amino acid sequence indicated that ABAP1 is a hydrophilic and basic protein possessing a number of potential glycosylation and praline hydroxylation sites. Notably, ABA1 has neither hydrophobic domains long enough to form membrane-spanning α-helices, nor is it a classical signal peptide. ABAP1 possesses a C-terminal WW protein interaction domain as shown in FIG. 1, which is characterized by two highly conserved tryptophan residues and a proline residue. The WW domain in ABAP1 generally fits the consensus sequence:
LxxGWtx6Gtx(Y/F)(Y/F)h(N/D)Hx(T/S)tT(T/S)tWxtPt
(where x=any amino acid, t=turn like or polar residue, and h=hydrophobic amino acid. Bold letters indicate invariant residues). Where there were deviations from the consensus sequence, more hydrophilic amino acids were substituted. FIG. 1 also shows the alignment of the ABAP1 WW domain with that of a flowering-time regulatory protein (FCA) from Arabidopsis (23), and the formin binding protein (FBP) of humans and mice.

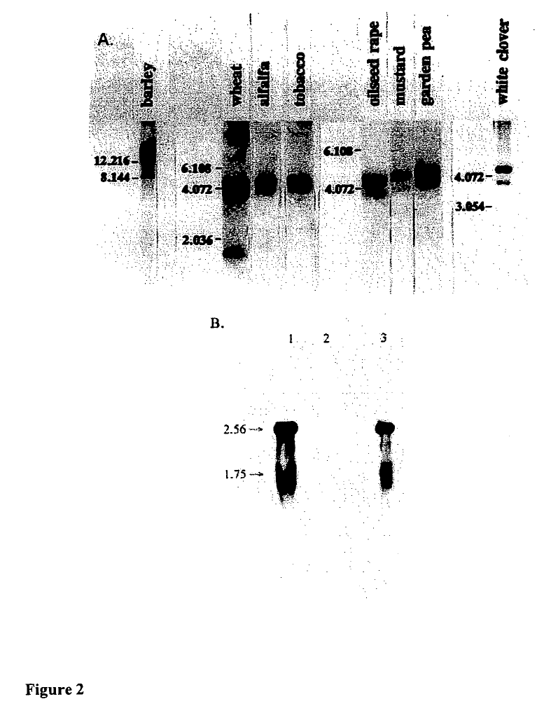

Genomic DNAs from various monocot and dicot plant species, including barley, wheat, alfalfa, tobacco, oilseed rape, mustard, garden pea, and white clover, contained ABAP1 positive genes as demonstrated by BamH1 digestion followed by Southern blot analysis as shown in FIG. 2. More than one ABAP1 positive band was detected in many of these plant species. Two prominent transcripts of approximately 2.6 and 1.8 kb were detected in Northern blot analysis of total RNA from barley aleurone, as shown in FIG. 2b, in keeping with the observations from the Southern analysis. While two transcripts could be observed in embryo and aleurone extracts, no hybridization signals were observed in RNA extracted from barley leaves. The 1.8 kb transcript corresponds to the size of ABAP1 cDNA.

ABAP1 Binds ABA

As shown in FIG. 3a, binding of ³H⁺-ABA to the purified ABAP1 protein linearly increased with increasing concentrations of ABAP1 in the assay medium. Heat denatured protein had no ABA binding activity as compared to ABAP1 (10 μg of each protein were used with buffer components). The ³H⁺-ABA binding to ABAP1 was sensitive to pH and maximum activity was achieved at pH 7.3, as shown in FIG. 3b.

The pH dependency of ABAP1 is consistent with earlier reports on the effect of pH on ABA function (11, 24) showing that ABA was more effective at neutral pH than either acidic or alkaline pH. Under drought stress, the compartmental pH of mesophyll, epidermis, guard cell, and phloem sap is shifted toward neutrality, suggesting that pH shifts under drought conditions might favour ABA binding to its receptor and so induce its function. The present results support this interpretation.

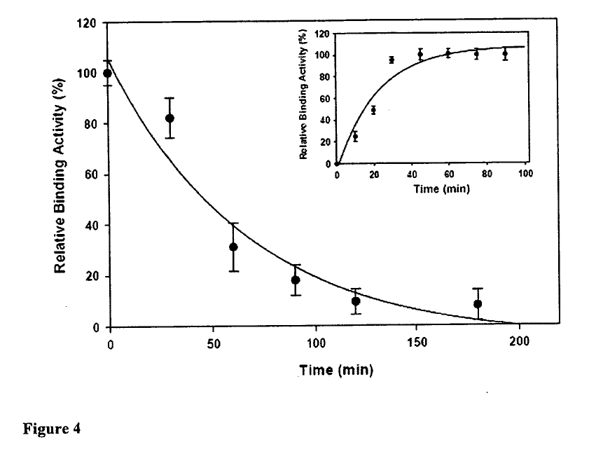

Association and dissociation kinetics of ³H⁺-ABA binding to ABAP1 are shown in FIG. 4, with the association reaction inset. The reaction was allowed to continue to equilibrium, at which point it was stopped by adding 100 μL of DCC. The dissociation experiment was then initiated by adding 5 μM unlabelled ABA. The specific binding capacity of ABAP1 was reversible.

The interaction of ³H⁺-ABA with ABAP1 was rapid and the maximum binding activity (˜0.7 mol ABA mol⁻¹ protein of total binding) remained stable for at least an additional 3 hours. Specific binding to ABAP1 was saturable with increasing amount of ³H⁺-ABA. Non-specific binding, as indicated by the lower line in FIG. 5a, was linear and always less than 10% of the total binding. When the data points from the saturable binding assays were transformed to a Scatchard plot, as shown in FIG. 5b, a linear function (r²=0.94) was observed. ABAP1 bound ABA at a ratio of approximately 0.8 mol ABA mol⁻¹ protein (FIG. 2b). The Scatchard plot showed one possible binding site for ABAP1 with a K_{d} calculated to be approximately 28 nM. As shown in FIG. 6, neither (−)-ABA nor trans-ABA compete for the ABA binding site on ABAP1. However, certain precursors of ABA, namely ABA aldehyde and ABA alcohol precursors did competitively inhibit binding of ABA to ABAP1 to some extent. The binding activity that was seen when (+) ABA-alcohol and (+) ABA-aldehyde were used indicates that the ABAP1 binding site tolerates, to some extent, alteration to the C-1 of ABA. Therefore, ABA C-1 may be altered without affecting binding to ABAP1. Both aldehyde and alcohol are ABA precursors that have previously been shown to have physiological activity.

ABA Binding Domain of ABAP1

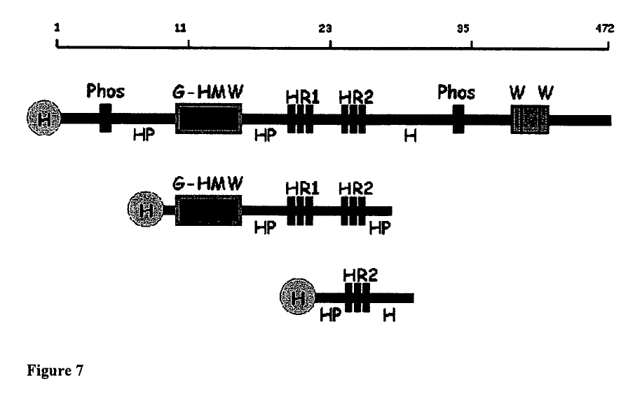

ABAP1 possesses conserved domains, including a high molecular weight elastomeric domain (G-HMW), hydrophobic regions flanked by highly hydrophilic platforms and a WW protein:protein interaction domain, as shown in FIG. 7. The G-HMW domain is an elastomeric domain because members of G-HMW containing proteins can withstand significant deformations without breaking under stress and return to the original conformation when the stress is removed.

Trypsin digests of ABAP1 resulted in three fragments approximately 26 kDa, 20 kDa, and 10 kDa. The two larger fragments retained the ability to bind AB2 antibodies whereas the smallest, 10 kDa fragment, had slight binding affinity to ABA. A 5 kDa 5′ hydrophilic end was removed from the largest 26 kDA fragment, resulting in a fragment that binds ABA at a similar molar ratio as full length ABAP1.

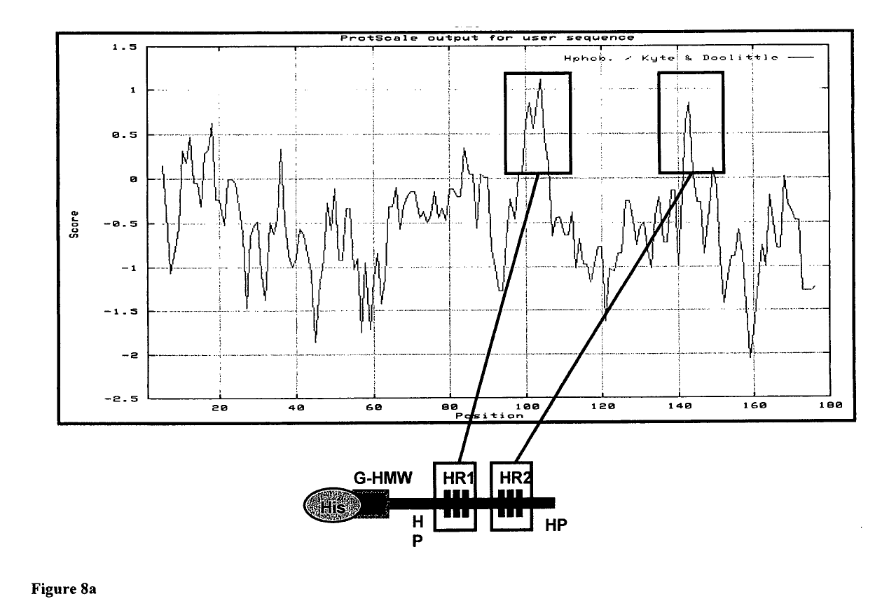

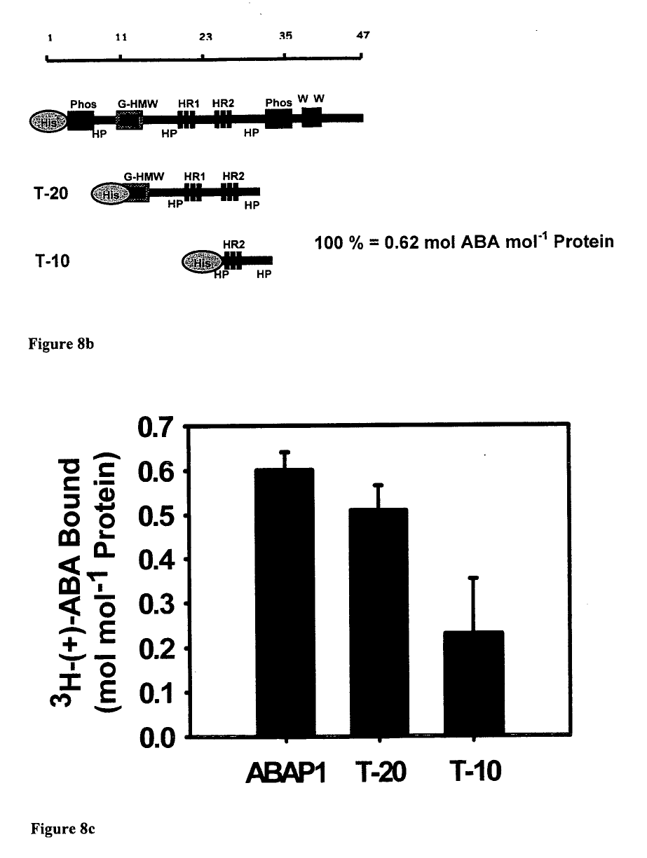

Hydrophobicity studies, shown in FIG. 8a, show that the 20 kDa fragment (T-20, as referenced in FIG. 8b) contains two hydrophobic regions, HR1 and HR2, flanked by a highly hydrophilic platform and the G-HMW domain. The shorter fragment of approximately 10 kDa (T-10) also contains the HR2 hydrophobic region.

In reference to FIG. 8c, ABA binding assays of all three peptides: ABAP1, T-20, and T-10, clearly shows that the ABA binding ability drastically decreased in the absence of the HR1 hydrophobic region. It can be inferred that the ABA binding motif require both the HR1 and the HR2 hydrophobic regions. Mutation analysis can be used to determine the specific residues involved in ABA binding.

Function of ABAP1

ABAP1 possesses a WW domain, which suggests that ABAP1 interacts with other proteins. The lack of a signal peptide, the hydrophilic nature of the protein and the lack of KDEL targeting peptide sequences, suggest that ABAP1 is a cytoplasmic protein, yet anti-idiotypic polyclonal antibodies (AB2), which recognized ABAP1 bound only to proteins associated with plasma and microsomal membrane fractions.

It is, therefore, understood that ABAP1 may be membrane-bound through its WW domain. The WW domains have been implicated in cell signalling and regulation, and are believed to act by recruiting proteins into signalling complexes. The domain interacts with proline-rich sequences and suggests that binding, in some instances, may require phosphorylation of a serine or threonine in the ligand (25), in an analogous fashion to SH2 domain binding to proteins containing phosphorylated tyrosine or 14-3-3 protein binding to phosphorylated serine residues in target proteins. Several of the identified proteins containing these domains regulate protein turnover in the cell and, in so doing, regulate other cellular events. Nedd4 is a ubiquitin protein ligase that binds a sodium channel protein, targeting it for turnover.

Unlike FCA protein, there is no evidence of RNA binding domains within ABAP1, making it unlikely that the protein would function as a post-transcriptional regulator.

ABAP1 Over Expression Activates eₘ (Early Methionin) Promoter

eₘ (early methionin) protein regulation is an another method to discover the role of ABAP1 in ABA signal transduction pathways achieved by studying the effects of an effector construct containing the full length ABAP1 in sense orientation under the control of an ubiquitin promoter on GUS (beta-glucuronidase) expression derived by the em protein promoter in the reporter construct.

The studies examined the effector and reporter constructs, at a 1:1 ratio, introduced to barley aleurone layers by gold particle bombardment. The bombardment consisted of two trials: the first trial was a bombardment of em promoter only; the second trial was a bombardment of eₘ promoters treated with ABAP1. The tissues were treated with different concentrations of ABA, at 0 μM, 5 μM, 10 μM, and 20 μM, and the resulting GUS activity observed.

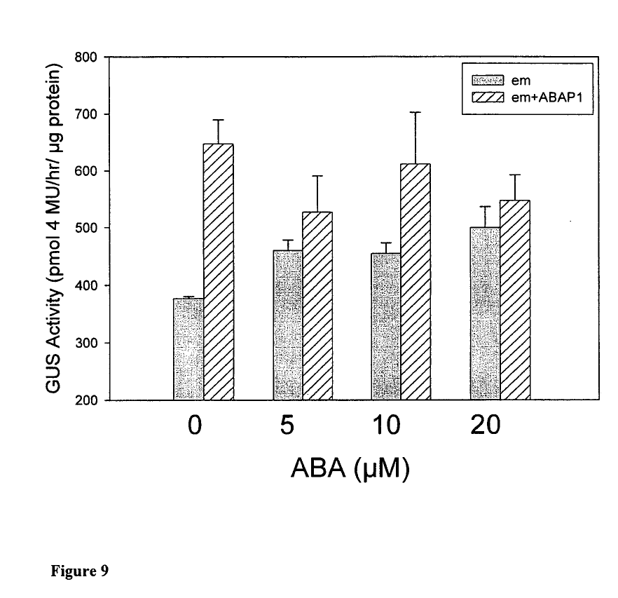

As shown in FIG. 9, GUS activity was twice fold when the aleurones were bombarded with eₘ proteins and ABAP1, as compared to the GUS activity when bombarded by eₘ proteins alone, without ABA treatment. However, under increasing concentrations of ABA, the difference in GUS activity between the aleurones bombarded with em alone and the aluerones bombarded with both em and ABAP1 are less significant.

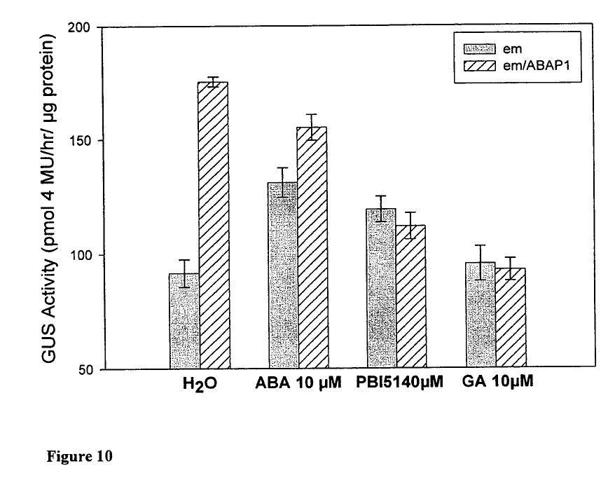

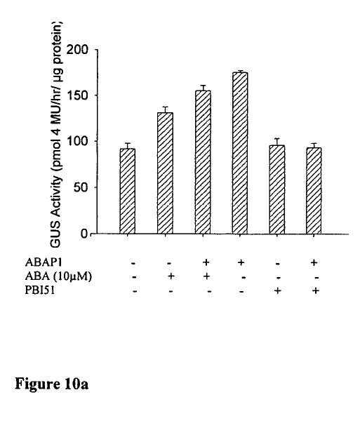

The high increase in eₘ promoter activity may have been due to high levels of endogenous ABA in the aleurones. By subjecting the aleurones to ABA, PBI51 (a competitive inhibitor of ABA) and GA, as shown in FIG. 10, the activation of the eₘ promoter by ABAP1 was reduced in the presence of PBI51 and GA. FIG. 10a summarizes the effect of ABAP1, ABA and PBI51 in varying combinations on GUS activity.

ABAP1 Inhibits α-Amylase Activity

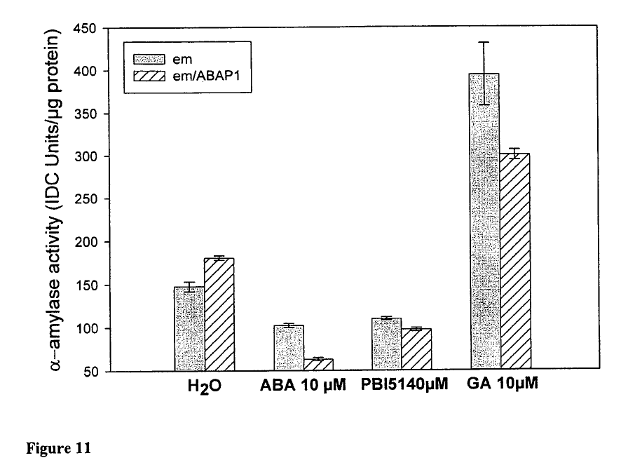

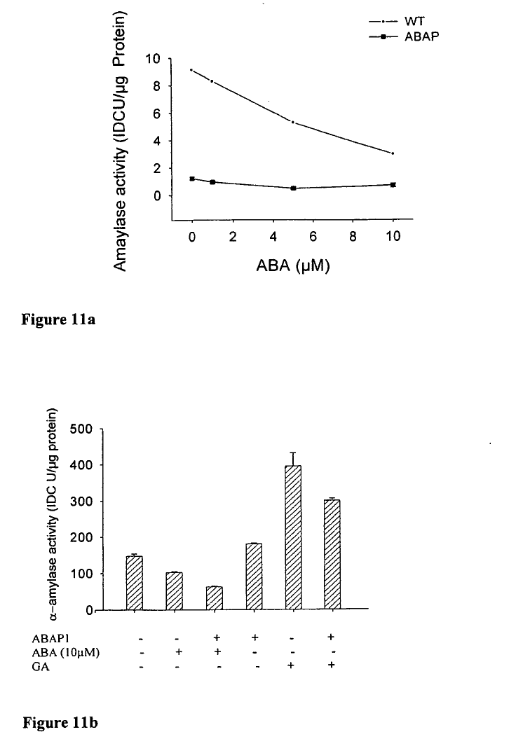

A similar experiment was conducted with α-amylase activity to confirm if ABAP1 is involved in another signal transduction pathway. α-amylase activity was measured after ABA, PBI51 and GA were added to the eₘ and ABAP1 bombarded barley aleurones. The results, as shown in FIG. 11, demonstrate the reduction of the α-amylase activity with the addition of ABAP1. α-amylase activity also decreased with the addition of PBI51, a competitive inhibitor of ABA, whereas the addition of non-competitive GA did not affect any reduction in α-amylase activity. Such results indicate that ABAP1 is a binding receptor for ABA.

FIG. 11a shows the affect of ABAP1 on α-amylase activity at varying ABA concentrations and FIG. 11b shows the affect of ABAP1, ABA and GA in varying concentrations on α-amylase activity.

ABAP1 Controls Seed Germination

To determine whether or not ABAP1 affects seed germination, mature embryo from two different barley lines (McLeod and Harrington) were bombarded with sense and anti-sense orientation of ABAP1. The embryos were subjected to different ABA treatments and the germination rate, plumule length, radical length and root numbers per embryo were measured for up to four days after bombardment.

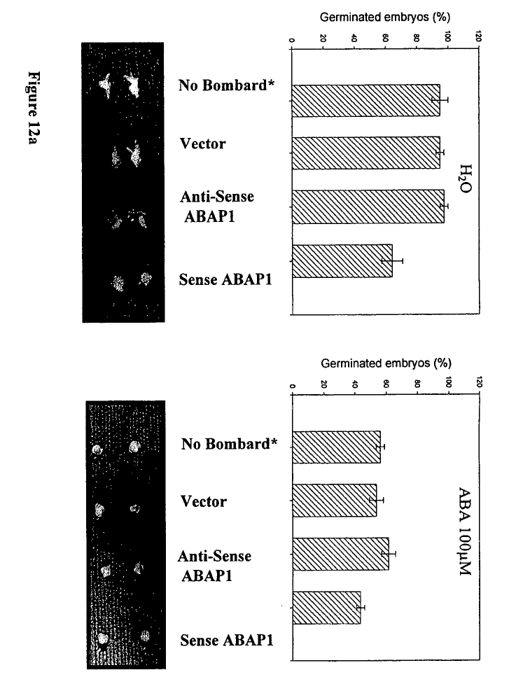

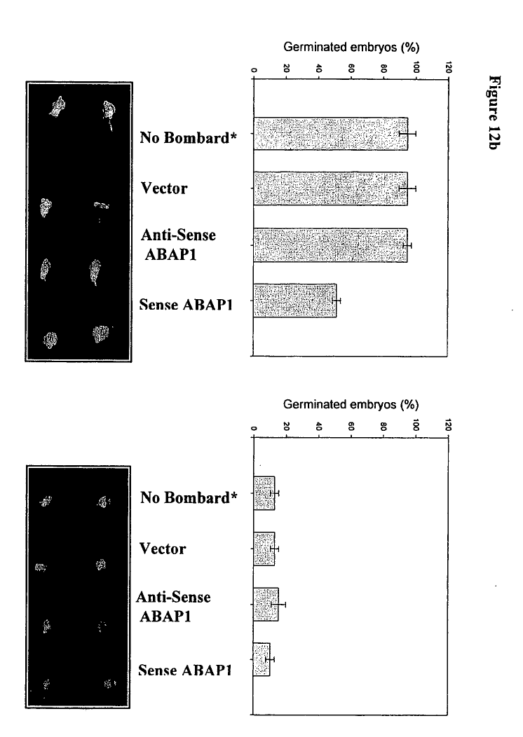

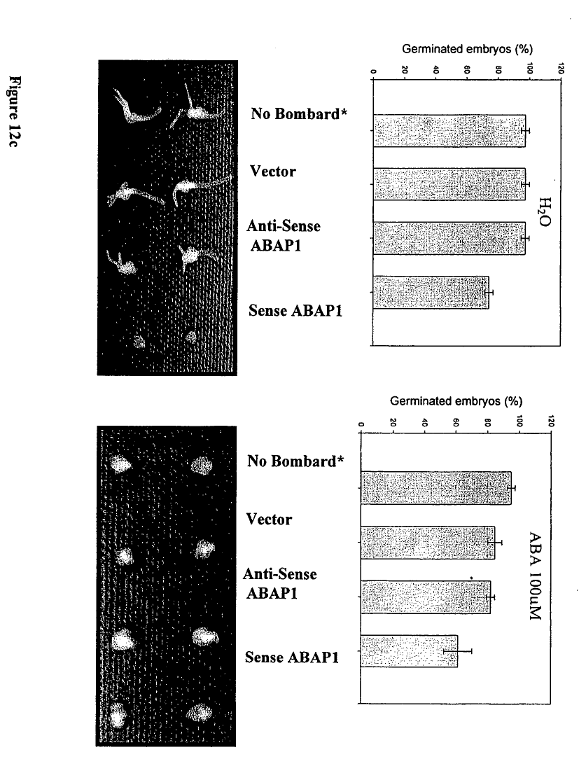

As shown in FIG. 12(a)-(c), the McLeod line of barley embryos showed a significantly lower germination rate in the presence of ABAP1, suggesting that ABAP1 inhibits seed germination. However, the germination rates did not seem to be affected by ABAP1 in the Harrington barley line, most likely due to the initial low level of ABAP1 transcripts previously noticed.

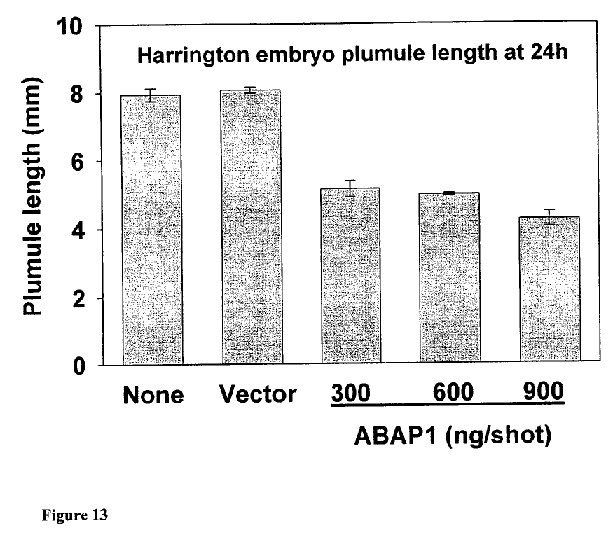

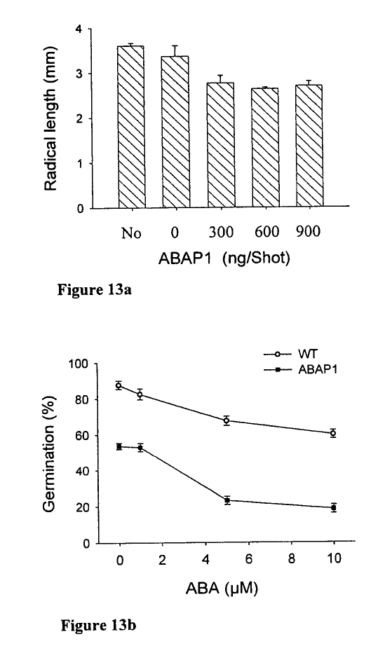

In the Harrington barley line, it was demonstrated that ABAP1 affects the plumule and radical growth of the embryos. FIGS. 13 and 13a show that the plumule and radical growth of barley embryos in the Harrington line, is significantly retarded in the presence of ABAP1.

FIG. 13b shows that the presence of ABAP1 significantly affects the germination of barley. This observation demonstrates that embryo development may be controlled in commercial processes such as barley malting where embryo development is not desired and where embryo development may otherwise reduce desired yields during such processes such as sugar and/or alcohol production.

Manipulation of Binding Sites

Methods to alter regulatory pathways that rely on the presence or absence of ABA and for inducing protective processes in a plant in which ABA or an ABA binding protein is administered to a plant are also described.

ABAP1 has Homology with FCA

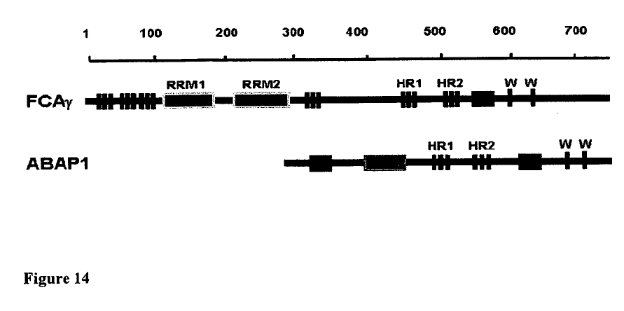

FCA is a plant specific RNA-binding protein having functions in the promotion/repression of flowering and the autoregulation of its own transcription. Hydrophobicity studies comparing both FCA and ABAP1, as shown in FIG. 14, shows that both proteins have the HR1 and HR2 hydrophobic regions required for ABA binding.

The following observations suggest that ABA binding sites may be conserved.

The pH dependency of FCA and ABAP1 are similar.

Similar molar binding ratios were obtained with ABAP1 and FCA. Furthermore, the FCA K_{d} for ABA of 19 nM is very close to the 28 nM obtained for ABAP1.

The specificity requirement (+ABA vs. −ABA) was also observed for both FCA and ABAP1.

These similarities suggest that the proteins coordinate with respect to their function in the presence of ABA.

It is likely that all ABA binding proteins will exhibit similar properties and may have homologous ABA binding sites. The conservation of these domains suggests homology to the degree such that FCA would bind ABA.

FCA Binds ABA

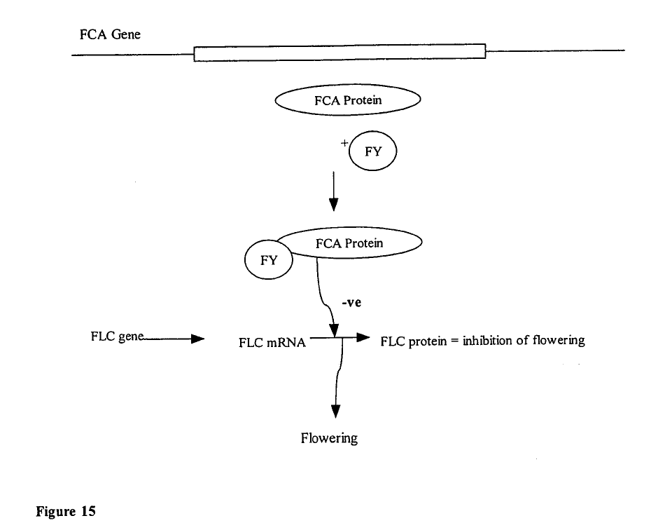

As shown in FIG. 15, the FLC gene is transcribed to mRNA, which is translated into FLC protein in order to repress flowering. When flowering is deemed necessary, the FCA gene is expressed to provide FCA protein. If FY is also present, an FCA-FY complex is formed through interaction of FCA WW region with FY. It has been suggested that the FCA-FY complex interferes with translation of FLC protein, thereby permitting flowering.

When ABA is present, ABA preferentially binds FCA, and displaces FY from FCA, if FY is present. The FCA-ABA complex does not inhibit translation of FLC protein, and therefore FLC protein will be produced to prevent flowering.

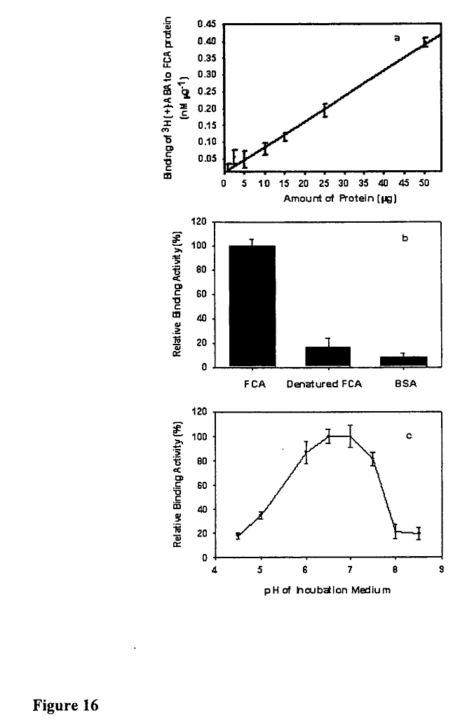

As shown in FIG. 16a, binding of ³H⁺-ABA to the purified FCA protein linearly increased with increasing concentrations of FCA in the assay medium. FIG. 16b shows that heat-denatured protein had no ABA binding activity as compared to FCA (10 μg of each protein plus buffer). As demonstrated in FIG. 16c, the ³H⁺-ABA binding to FCA was sensitive to pH and maximum activity was achieved over a pH range of 6.5 to 7.5 (100% binding activity corresponds to approximately 0.52 mol ABA mol⁻¹ protein).

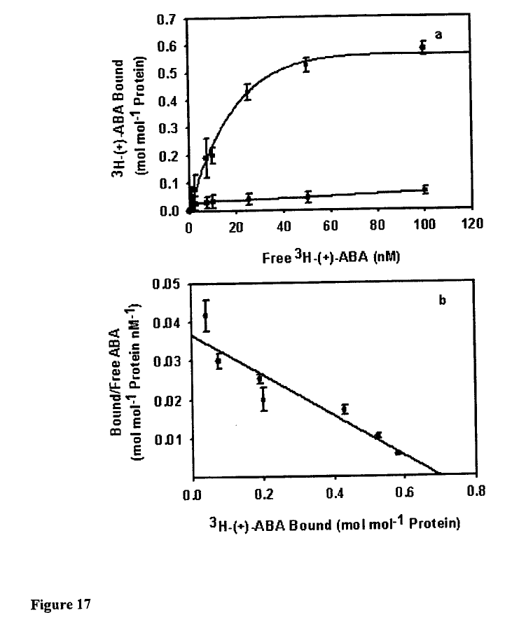

With reference to FIG. 17, FCA was incubated with increasing concentrations of ³H⁺-ABA in the absence of (total binding) or in the presence of 5 μM unlabelled (+)-ABA (non-specific binding). Specific binding of FCA with ABA is shown as the upper curve and non-specific binding is shown as the lower line. As shown, specific binding of ABA to purified FCA is saturable with increasing amounts of ³H⁺-ABA, and with non-specific binding less than 11% of the total binding. As shown in the Scatchard plot of FIG. 17b, a linear relationship (r²=0.88) was observed. When the first data point that represents a low concentration of ³H⁺-ABA in the incubation medium is excluded, linearity increased to r²=0.93, suggesting that FCA includes one ABA binding site. FCA bound ABA at a ratio of approximately 0.72 mol ABA mol⁻¹ protein, with an equilibrium dissociation constant (K_{d}) calculated to be approximately 19 nM.

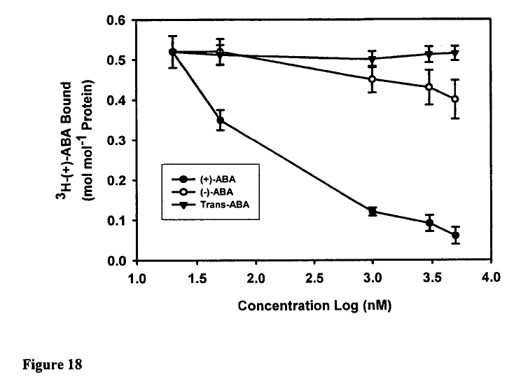

FCA binding kinetics meets the basic characteristics of an ABA receptor protein. The amount of ABA bound to FCA in the binding assays increased linearly with protein concentration but not with BSA or denatured FCA proteins, indicating that binding is specific for the native FCA protein. This specificity was also confirmed by using ABA analogs that might be expected to compete for the same binding site. Virtually no or very little displacement of ³H⁺-ABA binding was seen when (−)-ABA and trans-ABA was added to the binding assay in higher concentrations than ³H⁺-ABA (as shown in FIG. 18), an indication of the stereospecificity of FCA to the physiologically active (+)-ABA.

ABA Interferes with FCA/FY Interaction

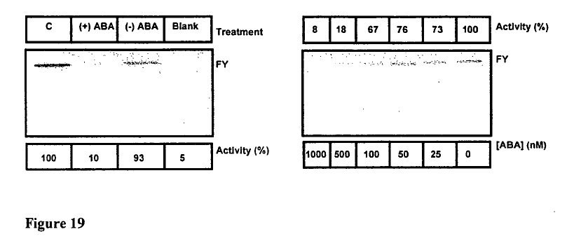

As shown in FIG. 19, when FCA is pre-bound with (+)-ABA, the interaction of FCA/FY was significantly inhibited. Inhibition of FCA/FY interaction by ABA was concentration-dependent and virtually no significant interaction between FCA and FY was observed at 1 μM ABA. Pre-incubation of FCA with (−)-ABA did not significantly inhibit FCA/FY interaction. Therefore, when ABA is bound to FCA, it is not easily displaced by FY.

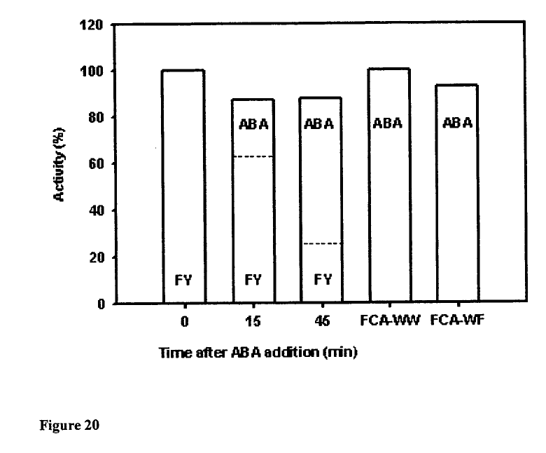

As shown in FIG. 20, when the binding study was repeated using FCA-WF, possessing a mutation in the second W that has been shown to prevent FCA/FY interaction (14), FCA-WF bound ³H⁺-ABA in virtually a similar ratio to the non-mutated FCA-WW protein. Therefore, although, FY binding to FCA requires an intact WW interaction domain, ABA binding does not require the FCA WW domain to be intact. ABA binding to FCA does, however, limit access by FY to the WW site and thus the FCA/FY complex is not favored. It is understood that ABA either causes a conformational change in the protein such that the WW site is not accessible, or ABA binds at a site adjacent to or overlapping at least a portion of the FY binding site. The former is likely, as it has been observed that the microenvironment of at least one W residue in ABAP1 becomes more hydrophobic upon binding of ABA, suggestive of a conformational change in the region of the WW domain. This indicates that alteration of the WW site or ABA binding site on the FCA protein would be possible to manipulate the effects of ABA on flowering and other related processes in plants.

Also with reference to FIG. 20, when a pre-formed FCA-FY complex was tested for its ability to bind ABA, the binding activity was initially low but significantly increased after 45 minutes as ABA displaced FY from the FCA protein. Therefore, ABA is capable of disrupting the FCA-FY complex. It is understood that ABA, by interfering with FCA/FY interaction, is inhibiting the downregulation of FLC, and thus plays a role for ABA in flowering that is likely to favour vegetative growth leading to a delay in flowering. This is in accordance with the physiological function of ABA in plants.

Method for Isolating ABA Binding Proteins

To date, efforts to isolate and characterize ABA receptors have been unsuccessful, despite the availability of antibodies and anti-idiotypic antibodies to ABA. Anti-idiotypic antibodies have been used to identify and isolate animal hormone receptors and to clone an ABA-inducible gene in barley aleurone (19). The present invention includes methods for the purification and characterization of ABA-binding proteins using AB2 antibodies.

Genetic analyses of mutants with altered responses to plant hormones have thus far failed to identify any putative ABA receptor (4). Attempts to study the early events of ABA action led to some success in describing proteins with different ABA-binding affinities that were prepared from cell extracts using conventional biochemical techniques (8-11). The major impediment to isolating ABA-binding proteins has been attributed to their low abundance relative to other proteins, their sensitivity, and their association with insoluble cell components.

The present recombinant protein approach is intended to circumvent these problems. Specifically, minimal amounts (0.5%) of SDS during cell lysis served to solubilize enough protein for purification, while maintaining catalytic activity. Unlike the case with most denaturants such as urea, detergent-solubilized proteins are often active and do not require a refolding step (21) as long as any excess of detergents is washed following lysis. To avoid further possible negative effects on protein and to maintain its stability, SDS was eliminated from all washing and elution steps and sucrose (250 mM) and glycerol (15-25% v/v) were supplemented to compensate for the lipid environment and to provide stability to preserve the protein functional conformation (21).

For protein storage, glycerol and sucrose were found to preserve protein activity after freezing. The catalytic activity has been confirmed by the ability of the purified ABAP1 protein to bind ABA at high mole to mole ratio relative to the denatured protein. The failure of ABAP1 to bind ABA with 1:1 ratio does not necessarily mean that part of the protein is denatured. It could rather mean that some of the binding sites are either unavailable (e.g., improper folding) for binding or inactivated due to various factors during purification. Furthermore, it should be noted that using detergents at low concentrations to solubilize receptor proteins is sometimes unavoidable, including for proteins with ABA binding affinities (e.g., CHAPS, 13; and Triton X-100, 15). This is likely because most receptor proteins are found to be on the plasma membranes and associated with hydrophobic domains.

Expression, Purification, and Immunodetection of ABA Binding Proteins

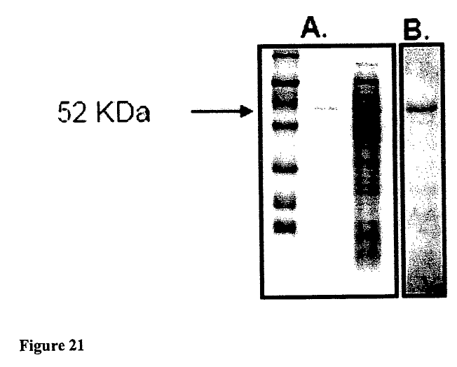

The ABAP1 protein was efficiently expressed under optimal induction and growth conditions of 1 mM IPTG at 37° C. However, the vast majority of the protein was associated with the insoluble fraction even when modifications were made to the expression system by either reducing temperature or IPTG concentration (data not shown). Because ABAP1 was difficult to obtain in the soluble fraction following cell lysis, due to its association with inclusion bodies, it was possible to solubilize enough protein by the addition of 0.5% SDS to carry out purification using the QIAexpress Purification System. Following purification, ABAP1 protein was purified and appeared as a single band on SDS-PAGE of apparent molecular weight of 52 kDa, as shown in FIG. 21a. When purified ABAP1 protein was probed with AB2 polyclonal anti-idiotypic antibodies, a single band of same molecular weight was detected (FIG. 13b).

FIG. 21 shows purification and immunodetection of ABAP1. In FIG. 21a, Coomassie blue-stained SDS-PAGE shows purified protein (middle lane), cell lysate (right lane), and markers (left lane). The calculated molecular weight of ABAP1 is 52 kDa. FIG. 21b shows that ABAP1 is detected by anti-idiotypic antibodies AB2.

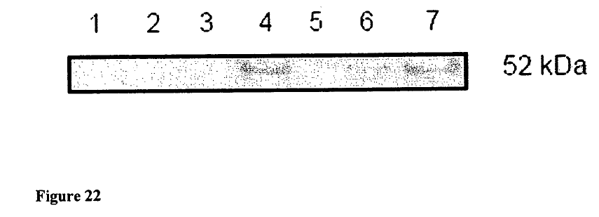

Membrane and cytosolic protein extracts from non ABA-treated and ABA-treated aleurone layers were separated by SDS-PAGE, blotted onto PVDF membrane and probed with AB2 antibodies. In FIG. 22, an immunoblot of subcellular fractions of barley aleurone layers using ABA AB2 antibodies is shown. Lane 1 and 2 indicate untreated and ABA-treated cytosolic fractions, respectively; lanes 3 and 4 indicate untreated and ABA-treated plasma membrane fractions, respectively; lanes 5 and 6 indicate untreated and ABA-treated microsomal fractions, respectively, and lane 7 contains ABAP1 as a positive control.

As is evident from FIG. 22, the AB2 polyclonal anti-idiotypic antibodies did not recognize any proteins from the cytosolic fractions of either non- or ABA-treated aleurones. AB2 antibodies detected, however, proteins with the appropriate molecular weight (i.e., 52 kDa) in the plasma membrane and microsomal fractions of ABA-treated aleurone layers. Although no bands were detected in the non ABA treated plasma membranes, a very faint band appeared in the microsomal fraction of the non ABA treated (difficult to be seen following scanning). The quality and purity of plasma membrane isolation were verified using appropriate marker enzyme assays as described in the experimental procedures (data not shown).

ABA45

ABAP1 possesses a WW domain to facilitate a protein:protein: interaction. A 35 kDa protein (termed ABA45) has been cloned from barley aleurones and shown to possess consensus domains that interact with WW domains. ABA45 includes a long transmembrane domain, suggesting association with aleurone plasma membranes. ABA45 also includes domains for SH3 interaction, and for binding kinases and phosphatases, suggesting a role in signalling.

One likely mechanism for ABA45 interaction with ABAP1 is to regulate signal transduction in the presence or absence of ABA (ie, if ABA is not present or is bound to FCA or ABAP1) and control time to flowering or seed dormancy or ripening.

### EXAMPLES

For examples 1 through 6, authentic ABA analogs were used for the stereospecificity studies and were provided by the National Research Council (NRC) of Canada—Saskatoon, Saskatchewan. All chemicals were purchased from Sigma unless otherwise stated.

### Example 1

### Expression and Purification of FCA Proteins

For ABA binding assays, FCA recombinant protein (the 3′ end of FCAγ possessing the WW domain) expressed in E. coli as a fusion protein^{S} with GST was purified. Seventy mL of LB culture media was infected by an overnight 10 mL culture of recombinant FCA- WW clone (plus 100 mg L⁻¹ ampicillin) and incubated for 30 minutes at 37° C. until OD₆₀₀ reached 0.5. The expression of FCA was induced by the addition of 1 mM IPTG and the culture was allowed to grow for 4 hours at 37° C. Following induction, the culture was centrifuged to pellet the cells and resuspended in 5 mL g⁻¹ PBST lysis buffer, pH 7.0 (10 mM Na₂H₂PO₄, 1.8 mM KH₂PO₄ 140 mM NaCl, 2.7 mM KCl, and 1% Triton X-100), left on ice for 15 minutes, freeze/thawed before sonication (6×10 seconds at 200-300 W with 10 second rests). Following centrifugation at 12,000 g at 4° C. for 20 minutes, the supernatant was mixed with 1 mL of pre-equilibrated (PBST) GST Affinity Resin (Stratagene) by shaking (200 rpm on circular rotator) at 4° C. for 60 minutes, loaded onto a column, washed 3 times with 3 ml PBST buffer each and then eluted with 4 volumes of 0.5 mL elution buffer (10 mM reduced glutathione (GSH) in 50 mM Tris-HCl, pH 8.0). Protein concentration was determined using the Bradford assay^{AA}.

Purification of the insoluble 5′ end of FCAγ possessing the RNA Recognition Motifs (FCA-RRM)^{S} was not carried out because preliminary ABA-binding assays using crude lysate from FCA-RRM did not show any ³H⁺-ABA binding and the protein was not characterized for ABA binding.

### Example 2

### ABA Binding Assays

Crude lysate and purified FCA protein were used to determine the ABA binding activity as described^{V}. Briefly, the incubation medium consisted of 12.5 mM Tris-HCl, pH 7.3 containing 50 nM ³H⁺-ABA (except when the kinetics of FCA was determined), and 10 μg purified FCA protein or the equivalent of 50 μg crude lysate. All binding assays were carried out at a final volume of 200 μL at 4° C. for 45 minutes. The mixture was then rapidly filtered through a nitrocellulose membrane, washed with 0.5× binding buffer, air dried and counted in a scintillation counter (Wallac 1414 WinSpectral v1.40). Heat denatured FCA protein was used to determine the protein nature of the FCA and BSA was used as a control. All binding studies were carried out using three different GST affinity chromatography protein purifications with triplicate assays for each purification. For the competitive asays, ABA analogs (−)-ABA and trans-ABA were added at the same time as ³H⁺-ABA at different concentrations (20-5000 nM). Specific binding was calculated by taking the difference for assays with only ³H⁺-ABA (total binding) and assays that also contained 5 μM (+)-ABA added at the same time as ³H⁺-ABA (non-specific binding). Binding was represented as the number of moles of ³H⁺-ABA per mole of FCA protein.

### Example 3

### GST Binding Assays of FCA-FY Interaction

All in vitro translation and GST pull-down assays were carried out as described by supplier's protocols (Promega, Madison, Wisc.) with modifications^{S} and as follows. For GST in vitro pull-down assays, 15 μL GST affinity resin was incubated with 250 μL FCA clear lysate, pelleted and the complex blocked and washed with IP buffer as described^{S}. For the determination of the amount of FCA bound to GST resin, the pellet was resuspended with 200 μL of 15 mM GSH to elute FCA and the supernatant was recovered by centrifugation. FY protein to be tested for interaction with the GST-FCA fusion protein was synthesized from a plasmid template and labeled with [³⁵S]-methionine using the T7 TNT coupled Transcription/Translation System (Promega). Twenty μL of FY labeled protein and 180 μL of interaction buffer (12.5 mM Tris-HCl, pH 7.3 containing 5 mM KCl, 1 mM MgCl₂, and 100 mM NaCl) were used to resuspend the GST:FCA after the final wash. The protein binding/interaction reaction was carried out for 90 minutes at 4° C. with continuous gentle mixing. The newly formed complex was then washed three times with 500 μL of IP wash buffer. After the final wash, the complex was resuspended, first with 10 μL of 15 mM GSH to facilitate the dissociation of interacted proteins from GST resin and then 10 μL of 2× SDS-PAGE sample buffer was added to the mixture and boiled for 5 minutes for complete elution of the proteins from the agarose beads. The beads were pelleted by centrifugation and supernatant was loaded on a 12% SDS-PAGE gel. The gel was dried and exposed to Kodak X-ray film for 18 hours at −70° C. and film was developed for the detection of labelled proteins.

### Example 4

### Effects of ABA on FCA/FY Complex

To test the effect of ABA on FCA/FY interaction, GST:FCA was incubated in interaction buffer in the presence of ABA FCA was bound with ABA for 30 minutes at which time the FY translated product was added to the incubation mixture. The interaction between FCA/FY was carried out in the presence of either (−)- or (+)-ABA in binding buffer as described above. Released proteins were separated on SDS-PAGE and labelled proteins were detected as described above. FCA-WW-FY was used as a control.

### Example 5

### Effects of WW Domain on ABA Binding

The GST:FCA-WW-FY interaction mixture was incubated for 90 minutes before 1 μM ³H⁺-ABA was added and the mixture pelletted, washed, and the dual activity for [³⁵S]-met-FY and ³H⁺-ABA were counted as described above. Time of incubation after ABA addition is shown and time 0 represents the GST:FCA-WW-FY activity before ABA addition.

Similarly, FCA-WF protein was used and binding assays were carried out as above. The activity of [³⁵S]-met-FY in the absence of ABA was similar to the control (time 0) at the time points shown and were not included in the figure. The FCA ³H⁺-ABA binding activity in the absence of FY reached approximately 50% saturation at 15 minutes and approximately 95% saturation at 45 minutes. Each data point represents triplicate assays and error bars represent standard deviation.

### Example 6

### Ability of ABA to Dissociate FCA/FY Complex

For the determination of FY dissociation from FCA-FY complex in the presence of ABA, the GST:FCA was collected by centrifugation either before or after ABA addition at the time points shown in figure legends, washed and resuspended in 100 μL IP buffer and dual activity for ³⁵S and ³H were counted simultaneously on a scintillation counter.

With respect to Examples 7 through 13, all chemicals were purchased from Sigma unless otherwise stated. Authentic ABA metabolites were obtained from the National Research Council (NRC) of Canada—Saskatoon, Saskatchewan. The AB2 antibodies were obtained from Dr. Shyam S. Mohapatra, University of South Florida, Division of Allergy and Immunology, Tampa, Fla. 33612, USA.

### Example 7

### Preparation of Aleurones and Plasma Membrane Isolation

Aleurone layers were prepared from mature barley seeds as described earlier (20). After incubation with 10 μM ABA for 24 hours, the aleurones were air dried and collected tissue was immediately frozen in liquid nitrogen, and either stored at −20° C. until used, or first ground to a fine powder in a pre-chilled mortar and pestle. Microsomal fractions were obtained by homogenizing ground tissue in homogenization buffer (100 mM MES buffer, pH 5.5 (5 mL g⁻¹) containing 250 mM sucrose, 3.0 mM EDTA, 10 mM KCl, 1.0 mM MgCl2, 0.5 mM phenylmethylsulfonyl fluoride (PMSF), and 1.0 mM freshly prepared DTT). The homogenate was filtered through four layers of cheesecloth and centrifuged for 10 minutes (15,000 g) at 4° C. The filtrate was centrifuged at 111,000 g for 60 minutes (4° C.) and the pellet, i.e., crude microsomal fraction (MF), used to isolate plasma membranes (PM) by dextranpolyethylene glycol aqueous two-phase partitioning. Cytosolic proteins were obtained from the 111,000 g supernatant (before phase partitioning) and protein concentration was measured using the Bradford protein assay. ATPase and NADPH-cytochrome C reductase activity were measured.

### Example 8

### Isolation of cDNA Clones

A λgt22A phage library was constructed using mRNA isolated from ABA-treated barley aleurone and a Superscript μgt22A cDNA construction kit (Invitrogen). The phage expression library was screened with the AB2 antibodies. Positive clones were isolated and the cDNA clones longer than 0.9 kb were subcloned into the NotI/SalI site of pBluescript SK vector. To obtain the full length cDNA for clone aba33, PCR amplification of aba33 positive phage from cDNA library was carried out using a primer designed from the 5′-end sequences of aba33 and a self designed primer for λgt22A. The cDNA was sequenced by the dideoxy procedure using the dsDNA cycle sequencing kit (Invitrogen) and the sequence is available on gene bank (Accession No. AF127388).

The coding region of the gene was amplified by RT-PCR with forward and reverse primers containing restriction enzyme linker sequences (ABA link F: CGGGATCCATGAATTCTCTTAGTGGGACTTA, ABA link R2: CTAGTCTAGATGCAGTCAACTTTTCCAAGAAC). The PCR product was ligated into the BamH1/Xba1 restriction site of the expression vector pPRoExHTb (Invitrogen) before being transformed into DH5α E. coli strain (Invitrogen). One clone (aba14) showing high expression of ABAP1 recombinant protein was selected for protein purification and characterization studies.

### Example 9

### Expression and Purification of ABAP1 Recombinant Protein

Expression and purification of ABAP1 that carry a carboxyl-terminal 6xHis-tag was carried out using the QIAexpress Purification System by affinity chromatography on Ni²⁺-NTA agarose columns (Qiagen) according to the manufacturer's instructions. Because the ABAP1 was highly insoluble due to the association with inclusion bodies, the following modifications to the manufacturer's protocol were carried out. Seventy mL of LB culture media was infected by an overnight 10 mL culture of recombinant aba14 clone (plus 100 mg L⁻¹ ampicillin) and incubated for 30 minutes at 37° C. until OD₆₀₀ reached 0.5. The expression of ABAP1 was induced by the addition of 1 mM IPTG and the culture was allowed to grow for 4 hours at 37° C. Following induction, the culture was centrifuged to pellet the cells and resuspended in 5 mL g⁻¹ lysis buffer, pH 8.0 (50 mM NaH₂PO₄, 300 mM NaCl, and 10 mM imidazole) that also included 15% glycine, 250 mM sucrose, and 0.5% (w/v) SDS, left on ice for 15 minutes, freeze/thawed before sonication (6×10 seconds with 10 second rests at 200-300 W). The addition of SDS was important to solubilize the protein, but it was later excluded from all subsequent purification steps, whereas sucrose was added to provide stability and to decrease the amount of detergent needed for solubilization.

Following centrifugation at 10,000 g at 4° C. for 25 minutes, the supernatant was mixed with 1 ml of 50% Ni²⁺-NTA agarose by shaking (200 rpm on rotary shaker) at 4° C. for 60 minutes before loaded on a column, washed with 8 mL washing buffer (50 mM NaH₂PO₄, 300 mM NaCl, 30 mM imidazole) and then eluted with elution buffer (50 mM NaH2PO4, 300 mM NaCl, 300 mM imidazole). Because the protein activity was maintained following purification, no refolding steps were needed (21), but the protein was supplemented with 15% glycerol and 250 mM sucrose to provide stability following purification. Although most binding assays were carried outusing a freshly prepared ABAP1, it was possible to store the protein with 25% glycerol (v/v) at −80° C. Protein concentration was determined using the Bradford assay.

### Example 10

### SDS-PAGE and Western Blot

The purified ABAP1 protein and membrane and cytosolic fractions (approximately 5 μg) were loaded on a discontinuous SDS-PAGE (15% separation gel) minigel system (BioRad) and separated according to the manufacturer's instructions. Proteins were transferred to polyvinylidine fluoride (PVDF) Millipore Immobilon-P membrane using a tank-blotting chamber (BioRad) and blots were blocked for 60 minutes at room temperature in blocking buffer (20 mM Tris-HCl, pH 7.5, 150 mM NaCl, 0.05 Tween 20 and 5% milk powder). After washing with washing buffer (TBS, 0.05% Tween 20), blots were incubated with AB2 antibodies (1:1000 dilution of 10 mg/mL), for 60 minutes at room temperature. Blots were washed 3× (twice for 10 minutes followed by a 15 minute wash) in washing buffer and subsequently incubated with secondary antibodies (1:1000 dilution, anti-mouse conjugated with alkaline phosphatase) for 60 minutes. Blots were washed as above and finally with _{dd}H2O (10 minutes). Blots were then immersed in staining buffer containing nitroblue tetrazolium (5% w/v) and bromochloroindolyl phosphate (5% w/v) in alkaline phosphatase buffer (100 mM Tris, pH 9.5, 100 mM NaCl, and 5 mM MgCl₂) for 10 minutes before the reaction was stopped by _{dd}H2O and blots were left to dry overnight at room temperature.

### Example 11

### Preparation of RNA, RNA Blotting and Northern Hybridization

Total RNA was isolated by using acid phenol procedures. Poly(A)+ mRNA was isolated using oligo dT-cellulose. The agarose gel electrophoresis of RNA followed methods described previously (22). Various amounts of mRNAs and 100 μg of total RNA (barley aleurone) were separated on a 1.5% denaturing agarose gel containing 2.2 M formaldehyde, 0.5 μg mL⁻¹ ethidium bromide and the separated RNAs were alkaline-transferred to Hybond N+ nylon membrane (Biosciences). The membranes were hybridized to an oligolabelled cDNA of clone ab33 under stringent conditions (6× SSC, 5× Denhardts, 2% SDS, 100 μg mL⁻¹ herring sperm DNA at 68° C.). The filters were finally washed in 0.2× SSC, 0.1% SDS at 65° C. and autoradiographed at −70° C. with an intensifying screen.

### Example 12

### Genomic DNA Isolation, Blotting, and Southern Hybridization

The genomic DNAs were prepared from different plants using a modified cetyl trimethylammonium bromide (CTAB) procedure as follows: the plant tissue was frozen in liquid nitrogen, ground into a fine powder and immediately placed in 1% hot CTAB buffer (1% CTAB in 100 mM Tris, pH 7.5, 10 mM EDTA, 400 mM NaCl, 0.14 M β-mecaptoethanol) and incubated at 60° C. for 1 hour. The genomic DNA was precipitated after phenol/chloroform extraction and RNase A digestion. The genomic DNA was digested with BamHI restriction enzyme. After separating the digested DNA in a 0.7% agarose gel and alkaline-transfer to Hybond N+ Nylon membranes, the blots were hybridized with the cDNA probe, ab33, under the conditions described above for northern hybridization.

### Example 13

### ABA Binding Assays

Purified ABAP1 protein was used to determine the ABA binding activity as described (15) with some modifications as follows. Generally, the incubation medium consisted of 25 mM Tris buffer, pH 7.3 (except when testing ABA binding at different pH) and 250 mM sucrose, 5 mM MgCl₂, 1 mM CaCl₂, 50 nM ³H⁺-ABA (except when the kinetics of ABAP1 was determined), and 10 μg ABAP1. Other additions or changes to the incubation system are discussed in the figure legends. All binding assays were carried out at a final volume of 150 μL at 4° C. for 1 hour. The mixture was then rapidly filtered through a nitrocellulose membrane, washed with 5 mL of cold 0.5× binding buffer by rapid filtration, dried in air and counted in a scintillation counter (Wallac 1414 WinSpectral v1.40). To ensure the efficiency of membrane washing and that only bound ³H⁺-ABA was counted, aliquots of the binding mixtures were mixed with a 100 μL of 0.5% (w/v) DCC (Dextran T70-coated charcoal) to remove any free ABA by adsorption. The DCC binding mixture was maintained for 15 minutes on ice before centrifugation to precipitate DCC. The resulted supernatant was then counted in a scintillation counter to determine the binding activity. Results from both were comparable with slight differences. Heat denatured ABAP1 protein was used to determine the protein nature of the ABAP1 and BSA was used as a control. All binding studies were carried out using three different protein purifications with triplicate assays for each purification. For the competitive assays, ABA analogs and precursors [(−) ABA, trans-ABA, PA, and DPA, ABA-aldehyde, ABA-alcohol, and ABA-GE] were added at the same time as ³H⁺-ABA at different concentrations (20-5000 nM). Specific binding (SB) was calculated by taking the difference for assays with only ³H⁺-ABA (total binding) and assays that also contain 5 μM (+) ABA added at the same time as ³H⁺-ABA (non-specific binding). Binding was represented as the number of moles of ³H⁺-ABA per mole of ABAP1 protein.

The above-described embodiments of the present invention are intended to be examples only. Alterations, modifications and variations may be effected to the particular embodiments by those of skill in the art without departing from the scope of the invention, which is defined by the claims appended hereto.

### REFERENCES

A. Kornneef, M., Alonso-Blanco, C., Peeters, A. J. M., & Soppe, W. Genetic control of flowering time in Arabidopsis. Annu. Rev. Plant Physiol. Plant Mol. Biol. 49, 345-370 (1998).
B. Kornneef, M., Alonso-Blanco, C., Blankestijn-de Vries, H., Hanhart, C. J., & Peeters, A. J. M. Genetic interactions among late-flowering mutants of Arabidopsis. Genetics 148, 885-892 (1998).
C. Levy, Y. Y., & Dean, C. The transition to flowering. Plant Cell 10, 1973-1989 (1998).
D. Simpson, G. G., Gendall, A. R., & Dean, C. When to switch to flowering. Annu. Rev. Cell Dev. Biol. 99, 519-550 (1999).
E. Araki, T. Transition from vegetative to reproductive phase. Curr. Opin. Plant Biol. 4, 63-68 (2001).
F. Mouradov, A., Cremer, F., & Coupland, G. Control of flowering time: interacting pathways as a basis for diversity. Plant Cell 14, S111-S130 (2002).
G. Henderson, I. R., Shindo, C., & Dean, C. The need for winter in the switch to flowering. Annu. Rev. Gen. 37, 371-392 (2003).
H. Samach, A., & Coupland, G. Time measurement and the control of flowering in plants. BioEssays 22, 3847 (2000).
I. Olszewski, N., Sun, T.-P., & Gubler, F. Gibberellin signaling: biosynthesis, catabolism, and response pathways. Plant Cell 14, S61-S80 (2002).
J. Michaels, S. D., & Amasino, R. M. FLOWERING LOCUS C encodes a novel MADS domain protein that acts as a repressor of flowering. Plant Cell 11, 949-956 (1999).
K. Michaels, S. D., & Amasino, R. M. Loss of FLOWERING LOCUS C activity eliminates the late-flowering phenotype of FRIGIDA and autonomous pathway mutations but not responsiveness to vernalization. Plant Cell 13, 935-941 (2001).
L. Simpson, G. G. The autonomous pathway: epigenetic and post-transcriptional gene regulation in the control of Arabidopsis flowering time. Curr. Opin. Plant Biol. 7, 570-574 (2004).
M. Sheldon, C. C., Rouse, D. T., Finnegan, E. J., Peacock, W. J., & Dennis, E. S. The molecular basis of vernalization: the central role of FLOWERING LOCUS C (FLC). Proc. Natl. Acad. Sci. USA 97, 3753-3758 (2000).
N. Michaels, S. D., & Amasino, R. M. Memories of winter: vernalization and the competence to flower. Plant Cell Environ. 23, 1145-1154 (2000).
O. Macknight, R., Bancroft, I., Page, T., Lister, C., Schmidt, R., Love, K., Westphal, L., Murphy, G., Sherson, S., Cobbett, C., & Dean, C. FCA, a gene controlling flowering time in Arabidopsis, encodes a protein containing RNA-binding domains. Cell 89, 737-745 (1997).
P. Macknight, R., Duroux, M., Laurie, R., Dijkwel, P., Simpson, G., & Dean, C. Functional significance of the alternative transcript processing of the Arabidopsis floral promoter FCA. Plant Cell 14, 877-888 (2002).
Q. Amasino, R. M. Flowering time: a pathway that begins at the 3′ end. Curr. Biol. 13, R670-R672 (2003).
R. Quesada, V., Macknight, R., Dean, C., & Simpson, G. G. Autoregulation of FCA pre-mRNA processing controls Arabidopsis flowering time. EMBO J. 22, 3152-3152 (2003).
S. Simpson, G. G., Dijkwel, P. P., Quesada, V., Henderson, I., & Dean, C. FY is an RNA 3′ end-processing factor that interacts with FCA to control the Arabidopsis floral transition. Cell 113, 777-787 (2003).
T. Page, T., Macknight, R., Yang, C.-H., & Dean, C. Genetic interactions of the Arabidopsis flowering time gene FCA, with genes regulating floral initiation. Plant J. 17, 231-239 (1999).
U. Blázquez, M. A., Green, R., Nilsson, O., Sussman, M. R. & Weigel, D. Gibberellins promote flowering of Arabidopsis by activating the LEAFY promoter. Plant Cell 10, 791-800 (1998).
V. Razem, F. A., Luo, M., Liu, J.-H., Abrams, S. R., & Hill, R. D. Purification and characterization of a barley aleurone abscisic acid-binding protein. J. Biol. Chem. 279, 9922-9929 (2004).
W. Zhang, D.-P., Wu, Z.-Y., Li, X.-Y., & Zhao, Z.-X. Purification and identification of a 42-kilodalton abscisic acid-specific-binding protein from epidermis of broad bean leaves. Plant Physiol. 128, 714-725 (2002).
X. Markarov, A. M. Causes of flowering of long-day potato species under short-day and cold-night conditions. Russ. J. Plant Physiol. 49, 465-469 (2002).
   1) Luo, M., Hill, R. D., and Mohapatra, S. S. (1992) Role of abscisic acid in plant responses to the environment. In: Plant responses to the environment: current topics in plant molecular biology. (Gresshoff, P. M., ed.). CRC Press, Roca Baton, Fla., pp. 147-165
   2) Hsu, Y. T., and Kao, C. H. (2003) Plant Cell Environ. 26, 867-874
   3) Mulholland, B. J., Taylor, I. B., Jackson, A. C., and Thompson, A. J. (2003) Environ. Exp. Bot. 50, 17-28
   4) Finkelstein, R. R., Gampala, S. S. L., and Rock C. D. (2002) Plant Cell S15- S45
   5) Leung, J., and Giraudat, J. (1998) Annu. Rev. Plant Physiol. Plant Mol. Biol. 49, 199-222
   6) Finkelstein, R. R. (1994) Plant J. 5, 765-771
   7) Yao, S.-G., Taketa, S., and Ichii, M. (2003) Plant Sci. 164, 971-978
   8) Hornberg, C., and Weiler, E. W. (1984) High-affinity binding sites for abscisic acid on the plasmalemma of Vicia faba guard cells. Nature 310, 321-324
   9) Pedron, J., Brault, M., Nake, C., and Miginiac E. (1998) Eur. J. Biochem. 252, 385-390
   10) Zhang, D.-P., Chen, S.-W., Peng, Y.-B., and Shen, Y.-Y. (2001) J. Exp. Bot. 52, 2097-2103
   11) Zhang, D.-P., Wu, Z.-Y., Li, X.-Y., and Zhao, Z.-X. (2002) Plant Physiol. 128, 714-725
   12) Yamazaki, D., Yoshida, S., Asami, T., and Kuchitsu, K. (2003) Plant J. 35, 129-139
   13) Leung, J., Bouvier-Durand, M., Morris, P., Guerrier, D., Chefdor, F., and Giraudat, J. (1994) Science 264, 1448-1452
   14) Meyer, K., Leube, M. P., and Grill, E. (1994) Science 264, 1452-1455
   15) x20-Wu, Y., Sanchez, J. P., Lopez-Molina, L., Himmelbach, A., Grill, E., and Chua, N.-H. (2003) Plant J. 34, 307-315
   16) Meinhard, M., and Grill, E. (2001) FEBS Lett. 508, 443-446
   17) Kwak, J. M., Mori, I. C., Pei, Z.-M., Leonhardt, N., Torres, M. A., Dangl, J. L., Bloom, R. E., Bodde, S., Jone, J. D. G., and Schroeder, J. I. (2003) EMBO J. 22, 2623-2633
   18) Jaradat, Z. W., and Marquardt, R. R. (2001) Food Agr. Immun. 13, 241-253
   19) Liu, J.-H., Luo, M., Cheng, K.-J., Mohapatra, S. S., and Hill, R. D. (1999) J. Exp. Bot. 50, 727-728
   20) Hill, R. D., Durnin, D., Nelson, L. A. K., Abrams, G. D., Gusta, L. V., and Abrams, S. R. (1992) Seed Sci. Res. 2, 207-214
   21) Villaverde, A., and Mar Carriö, M. (2003) Biotech. Lett. 25, 1385-1395
   22) Luo, M., Liu, J.-H., Mohapatra, S., Hill, R. D., and Mohapatra, S. S. (1992) J. Biol. Chem. 267, 15367-15374
   23) Macknight, R., Bancroft, I., Page, T., Lister, C., Schmidt, R., Love, K., Westpal, L., Murphy, G., Sherson, S., Cobbett, C., and Dean, C. (1997) Cell 89, 737-745
   24) Anderson, B. E., Ward, J. M., and Schroeder, J. I. (1994) Plant Physiol. 104, 1177-1184
   25) Lu, P.-J., Zhou, X. Z., Shen, M., and Lu, K. P. (1999) Science 283, 1325-1328
   26) Bonetta, D., and McCourt, P. (1998) Trend. Plant Sci. 3, 231-235.
   27) Jeannette, E., Rona, J.-P., Bardat, F., Cornel, D., Sotta, B., and Miginiac, E. (1999) Plant J. 18, 13-22
   28) Ghelis, T., Dellis, O., Jeannette, E., Bardat, F., Cornel, D., Miginiac, E., Rona, J.-P., and Sotta, B. (2000) FEBS Lett. 474, 43-47

What is claimed is:

1. A method of regulating the expression of proteins in seed development comprising the step of introducing an effective amount of ABAP1 or an operative fragment thereof into a developing seed.

2. A method of regulating seed germination comprising the step of introducing an effective amount of ABAP1 or an operative fragment thereof into a seed.

3. A method as in claim 1 wherein the ABAP1 is introduced into the aleurone of a seed.

4. A method as in claim 1 wherein the ABAP1 is introduced into the embryo of a seed.

5. A method as in claim 2 wherein the ABAP1 is introduced into the aleurone of a seed.

6. A method as in claim 2 wherein the ABAP1 is introduced into the embryo of a seed.

7. A method as in claim 1 wherein the step includes regulating the eₘ promoter in a plant seed by introducing an effective amount of ABAP1 or an operative fragment thereof into the seed.

8. A method as claim 1 wherein the method is conducted in the presence of abscisic acid (ABA).

9. A method as claim 2 wherein the method is conducted in the presence of abscisic acid (ABA).

10. A method for synergistically regulating the expression of proteins in seed development comprising the step of introducing an effective amount of ABAP1 or an operative fragment thereof and abscisic acid (ABA) into a developing seed.

11. An ABAP1 fragment retaining abscisic acid (ABA) binding capability.

12. An ABAP1 fragment as in claim 11 wherein the fragment is 10 kDa or larger characterized by a hydrophobic region HR2.

13. An ABAP1 fragment as in claim 11 wherein the fragment is 21 kDa or larger characterized by two hydrophobic regions, HR1 and HR2.

14. An ABAP1 fragment retaining abscisic acid (ABA) binding capability formed by trypsin digestion of ABAP1.

15. A method of modulating abscisic acid (ABA)-mediated signal transduction comprising the step of introducing an effective amount of ABAP1 or an operative fragment thereof or ABA or mixtures thereof to regulate plant flowering, germination and dormancy.

16. A method as in claim 15 comprising the step of introducing an effective amount of ABAP1 or an operative fragment thereof to the plant to promote plant flowering.

17. A method as in claim 15 comprising the step of introducing an effective amount of ABA to the plant to inhibit plant flowering.

18. A method as in claim 15 wherein the applied concentration of ABA is 0-1000 nM.

19. A method as in claim 17 wherein the concentration of ABA is greater than 0 and plant flowering is inhibited.

20. A method of isolating and purifying ABAP1 comprising the steps of:
a) infecting a recombinant clone;
b) inducing over expression of ABAP1; and,
c) isolating and purifying ABAP1.

21. A method as in claim 20 wherein aba14 recombinant clone is used to express ABAP1.

22. A method as in claim 20 wherein a aba14 recombinant clone is infected to express ABAP1

23. A method as in claim 20 wherein ABAP1 expression is induced in step b) by the addition of IPTG.
